(19) United States
(12) Patent Application Publication
(10) Pub. No.: US 2021/0264537 A1
(43) Pub. Date: Aug. 26, 2021

(54) SYSTEMS AND COMPUTER-IMPLEMENTED METHODS TO DYNAMICALLY ADJUST IN REAL-TIME A DEDUCTIBLE, PREMIUM, OR INCENTIVE FOR AN INSURANCE PLAN

(71) Applicant: The Karis Group, Austin, TX (US)

(72) Inventor: Matthew Dale, Austin, TX (US)

(21) Appl. No.: 17/249,333

(22) Filed: Feb. 26, 2021

Related U.S. Application Data
(60) Provisional application No. 62/981,799, filed on Feb. 26, 2020.

(51) Int. Cl. G06Q 40/08; G16H 40/20

(52) U.S. Cl. CPC G06Q 40/08; G16H 40/20

(57) ABSTRACT

A system and method for dynamically adjusting in real-time a deductible, premium, or incentive for an insurance plan is provided. The system can include a server computing device and a provider computing device connected via a communications network. The server computing device may be coupled to one or more mobile user devices associated with insureds. The server computing device can execute an online tool operable to receive price updates for procedures from providers through the provider computing device and update the price for the procedure offered by the provider in real-time on a mobile application displayed on the mobile user devices. The online tool can further be operable to dynamically adjust in real-time a premium, deductible, or incentive for an insurance plan of each insured based on the updated price for the procedure, and display the premium, deductible, or incentive on the mobile application associated with the mobile user device.

### CROSS-REFERENCE TO RELATED APPLICATIONS

This application claims priority to U.S. Provisional Patent Application Ser. No. 62/981,799, filed on Feb. 26, 2020, to Matthew Dale, entitled “Systems and Computer-Implemented Methods to Dynamically Adjust in Real-Time a Deductible, Premium, or Incentive for an Insurance Plan,” currently pending, the entire disclosure of which is incorporated herein by reference.

### FIELD OF INVENTION

The present invention relates generally to insurance technology and, more particularly, to systems, machines, computer-implemented methods, and non-transitory computer media having computer program instructions stored thereon for dynamically adjusting in real-time a deductible, premium, or incentive for an insurance plan.

### BACKGROUND OF THE INVENTION

It is a common frustration of insurance companies and healthcare sharing organizations that patients do not always choose the lowest cost provider. For example, if a patient has a $500 deductible, the patient will pay the same price for a procedure when Hospital A offers it for $50,000, Hospital B offers it for $75,000, and Hospital C offers it for $100,000. Because the patient pays $500 regardless, the patient does not care which hospital they select. And because patients often mistakenly equate cost of care with quality of care, the patient will frequently pick the most expensive hospital under the assumption that it is the best one. Thus, there is a need for encouraging patients to use the medical provider having the lowest cost.

### BRIEF SUMMARY OF THE INVENTION

The present invention is directed generally to a system and method for dynamically adjusting in real-time a deductible, premium, or incentive for an insurance plan. The system and method of the present invention can enable service providers (e.g., healthcare provider, automobile repair provider, etc.) to update or change the pricing of procedures or services offered by the service provider in real-time and adjust a deductible, premium, or incentive for an insurance plan of insureds (e.g., patients, automobile owners, etc.). The system and method of the present invention may further have the objective of continually incentivizing insureds to select the lowest cost service provider by updating incentives dynamically and in real time as a service provider changes its pricing.

According to one embodiment, the system can include at least one server computing device, at least one healthcare provider computing device, and at least one mobile user device, all of which can be communicatively and/or electrically coupled via a communications network, such as the Internet. The system can further include an online tool executing on the at least one server computing device. The online tool can be operable to receive price changes to procedures or services offered by a healthcare provider associated with the healthcare provider computing device, update the price in real-time for potential patients or insureds, and transmit the price change to a mobile application executing on the mobile user devices associated with the one or more patients or insureds. The online tool can further be operable to dynamically adjust in real-time a premium, deductible, or incentive for an insurance plan of each potential patient or insured based on the changed price for the healthcare procedure or service, and transmit data comprising the adjusted premium, deductible, or incentive to the mobile application executing on the mobile user devices associated with the one or more patients or insureds.

The system can include menu builder (displayed via a graphical user interface) executable on the healthcare provider computing device that can allow the healthcare provider to create procedures or services and a price associated therewith. The menu builder can further enable the healthcare provider to change the price of a procedure or service at any time and the system (via the online tool) can update the changed price in real-time.

The system can include a mobile application executable on the mobile user devices associated with the patients or insureds and operable receive a price change from the online tool associated with a procedure or service offered by the healthcare provider, receive data associated the adjusted premium, deductible, or incentive from the online tool, and display (e.g., via a graphical user interface) the updated price and/or the adjusted premium, deductible, or incentive.

The method of the present invention can be configured for dynamically adjusting in real-time a deductible, premium, or incentive for an insurance plan. The method can include the steps of: receiving a price change of a healthcare procedure from a healthcare provider through the healthcare provider computing device; transmitting the price change of the healthcare procedure to the online tool executing on the server computing device; and dynamically adjusting in real-time a deductible, premium, or incentive for an insurance plan of at least one patient covering the healthcare procedure based on the price change.

According to one embodiment, the method can include the step of transmitting data comprising the price change from the online tool to the mobile application executing on the mobile user device of the at least one patient. According to one embodiment, the method can include the step of transmitting data comprising the dynamically adjusted premium, deductible, or incentive from the online tool to the mobile application executing on the mobile user device of the at least one patient.

Other aspects and advantages of the present invention will be apparent from the following detailed description of the preferred embodiments.

### DESCRIPTION OF THE SEVERAL VIEWS OF THE DRAWINGS

In the accompanying drawings, which form a part of the specification and are to be read in conjunction therewith in which like reference numerals are used to indicate like or similar parts in the various views:

FIG. 1 is a schematic diagram of a system for dynamically adjusting in real-time a deductible, premium, or incentive for an insurance plan in accordance with one embodiment of the present invention;

FIG. 2 is a schematic flow chart of a method for dynamically adjusting in real-time a deductible, premium, or incentive for an insurance plan in accordance with one embodiment of the present invention;

FIG. 3 is a schematic diagram of a mobile application graphical user interface displaying real-time price updates on a mobile user device used with the system of FIG. 1 in accordance with one embodiment of the present invention;

FIG. 4 is a schematic diagram view of a mobile application graphical user interface displaying an adjusted premium, deductible, or incentive on a mobile user device used with the system of FIG. 1 in accordance with one embodiment of the present invention; and

FIG. 5 is a schematic block diagram of a computer system for executing the system of FIG. 1 and the method of FIG. 2 in accordance with one embodiment of the present invention.

### DETAILED DESCRIPTION OF THE INVENTION

The invention will now be described with reference to the drawing figures, in which like reference numerals refer to like parts throughout. For purposes of clarity in illustrating the characteristics of the present invention, proportional relationships of the elements have not necessarily been maintained in the drawing figures. It will be understood that any dimensions included in herein are simply provided as examples and dimensions other than those provided therein are also within the scope of the invention.

The following detailed description of the invention references specific embodiments in which the invention can be practiced. The embodiments are intended to describe aspects of the invention in sufficient detail to enable those skilled in the art to practice the invention. Other embodiments can be utilized and changes can be made without departing from the scope of the present invention.

One exemplary objective of an embodiment of the present invention is to continually incentivize insureds (e.g., patients, automobile owners, etc.) to select the lowest cost service provider (e.g., healthcare provider, automobile repair provider, etc.) by updating incentives dynamically and in real time as a service provider changes its pricing. Although embodiments described herein are in terms of health insurance, those skilled in the art will appreciate that aspects of the invention can be applied to other industries or fields, including but not limited to dental insurance, disability insurance, life insurance, pet insurance, homeowners or renters insurance, flood insurance, automobile insurance, agricultural insurance, and travel insurance.

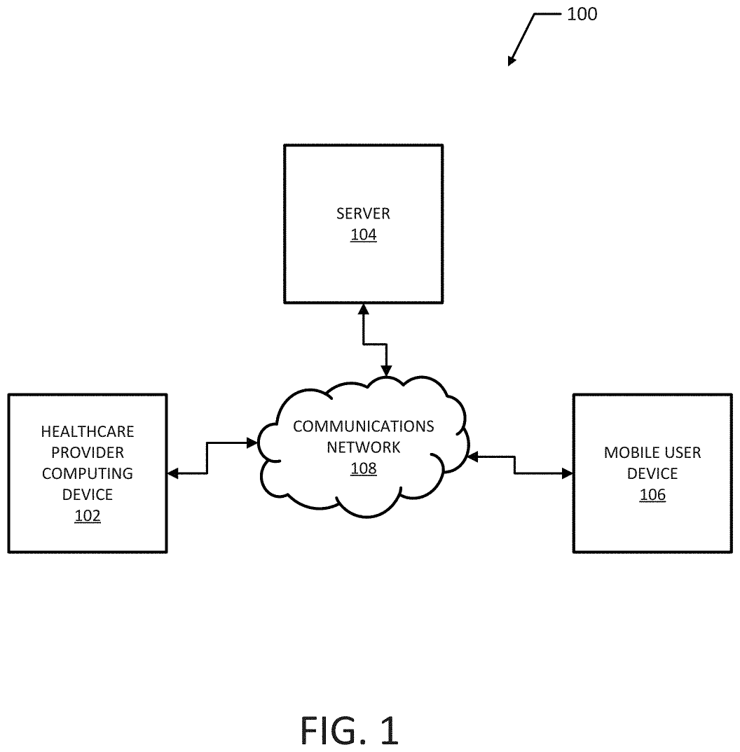

FIG. 1 illustrates a dynamic adjusting system 100. In an embodiment, the dynamic adjusting system 100 can be operable to dynamically adjust in real-time a deductible, premium, or incentive for an insurance plan. In the embodiment shown in FIG. 1, the dynamic adjusting system 100 includes at least one healthcare provider computing device 102, at least one server 104, and at least one mobile user device 106 that are communicatively and/or electrically coupled via a communications network 108, such as the Internet.

The healthcare provider computing device 102 can be operable to provide a menu builder (e.g., via a graphical user interface) to a user (e.g., a medical provider, etc.) to allow the user to create procedures on which prices for healthcare procedures can be updated. In an embodiment, the menu builder operates like the backend of an e-commerce site. For example, if a provider offers knee replacement, the provider can list the price it charges for the procedure and change that price at any time. The healthcare provider computing device 102 can be further operable to provide access to an online tool executing on the server 104 and transmit data comprising price changes to the online tool executing on the server.

The server 104 can be operable to execute an online tool that manages data of healthcare providers and healthcare patients who have insurance plans. The online tool executing on the server 104 can be operable to receive data comprising price changes from the healthcare provider computing device 102, update the price in real-time for potential patients, transmit data comprising the updated price to a mobile application executing on the mobile user devices 106, execute one or more algorithms with the changed price as an input to dynamically adjust in real-time a premium, deductible, or incentive for an insurance plan of each potential patient based on the changed price for the healthcare procedure, and transmit data comprising the adjusted premium, deductible, or incentive to a mobile application executing on the mobile user devices 106.

The mobile user device 106 can be operable to receive data comprising the updated price from the online tool executing on the server 104, receive data comprising the adjusted premium, deductible, or incentive from the online tool executing on the server 104, and execute a mobile application for displaying (e.g., via a graphical user interface) the updated price and/or the adjusted premium, deductible, or incentive.

The communications network 108 can be capable of facilitating the exchange of data among computing devices, including the healthcare provider computing device 102, the server 104, and the mobile user device 106. The communications network 108 can include a local area network (LAN) that is connectable to other communications networks, including other LANs, wide area networks (WANs), and/or portions of the Internet or an intranet. The communications network 108 can also include any communications network that facilitates the exchange of data, such as those that operate according to the IEEE 802.3 (e.g., Ethernet®) and/or the IEEE 802.11 (e.g., Wi-Fi®) protocols, for example. In other embodiments, the communications network 108 includes any medium that allows data to be physically transferred through serial or parallel communication channels (e.g., copper wire, optical fiber, computer bus, wireless communication channel, etc.).

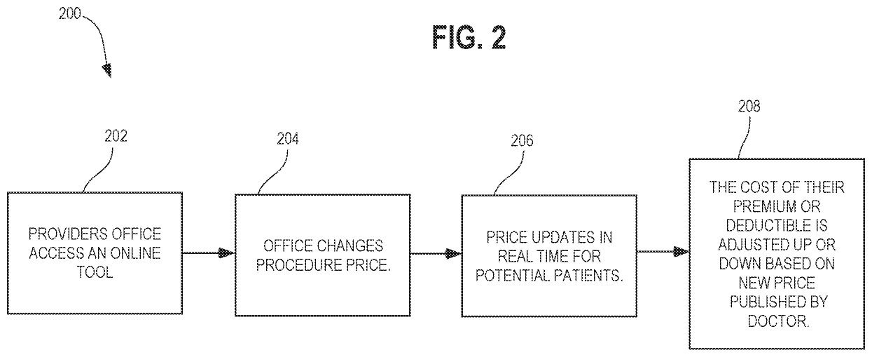

FIG. 2 illustrates a method 200 for dynamically adjusting in real-time a deductible, premium, or incentive for an insurance plan. At 202, a user of the healthcare provider computing device 102 accesses an online tool executing on the server 104. For example, the user of the healthcare provider computing device 102 can access the online tool on the server 104 via the communications network 108. In an embodiment, the dynamic adjusting system 100 includes a “menu builder” that allows medical providers (e.g., a user of healthcare provider computing device 102, etc.) to create procedures on which they can update prices. The menu builder can operate like the backend of an e-commerce site. For example, if a provider offers a knee replacement procedure, the provider can list the price it charges for the procedure and change the price at any time. The price that the provider enters automatically pushes the new prices to a backend server (e.g., server 104) that runs the new price through an algorithm to change in real-time the price that is pushed through to a mobile application used by patients, as further described herein. At 204, the user of the healthcare provider computing device 102 changes a price for a healthcare procedure. For example, the online tool executing on the server 104 can utilize a database stored on the server 104 that includes healthcare procedures (e.g., knee replacement, etc.) and an associated price for the healthcare procedure charged by the healthcare provider associated with the healthcare provider computing device 102 and the user. Changing the price for a healthcare procedure can include altering existing pricing data stored in the database. At 206, the online tool executing on the server 104 updates the price in real-time for potential patients. In an embodiment, step 206 can include altering data in a database stored on the server 104 to reflect the updated price. Additionally or alternatively, step 206 can include transmitting data comprising the updated price to a mobile application executing on the mobile user devices 106. The mobile application executing on the mobile user devices 106 can generate a notification of the updated price, display the updated price on a graphical user interface (GUI), or the like. At 208, the online tool executing on the server 104 dynamically adjusts in real-time a premium, deductible, or incentive for an insurance plan of each potential patient based on the changed price for the healthcare procedure. In an embodiment, step 208 can include altering data in a database stored on the server 104 to reflect the adjusted premium, deductible, or incentive. Additionally or alternatively, step 208 can include transmitting data comprising the adjusted premium, deductible, or incentive to a mobile application executing on the mobile user devices 106. The mobile application executing on the mobile user devices 106 can generate notifications of the adjusted premium, deductible, or incentive, display the adjusted premium, deductible, or incentive on a GUI, or the like. Accordingly, the dynamic adjusting system 100 takes the latest pricing data from medical providers and continually updates a consumer-facing (i.e., patient-facing) application to always incentivize patients to select the lowest cost healthcare provider.

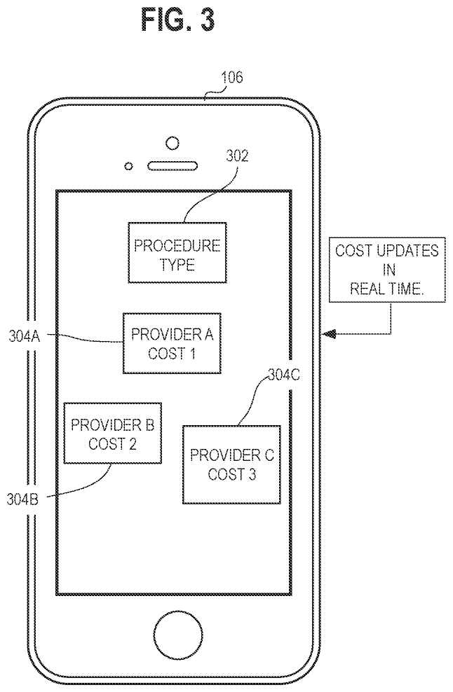

FIG. 3 illustrates an exemplary GUI of the mobile application executing on the mobile user devices 106 displaying price updates in real-time. The exemplary GUI includes a procedure type element 302 and a plurality of provider elements 304a, 304b, 304c. The procedure type element 302 displays a name or identifier of the procedure (e.g., “knee replacement”, etc.). Each provider element 304a, 304b, 304c displays a name or identifier of the provider (e.g., “Provider A”, etc.) and the price the provider charges for the procedure displayed in the procedure type element 302 (i.e., the cost of the procedure as performed by that provider). Although the exemplary GUI illustrated in FIG. 3 includes three provider elements 304a, 304b, 304c, those skilled in the art will appreciate that the GUI may include any number of provider elements.

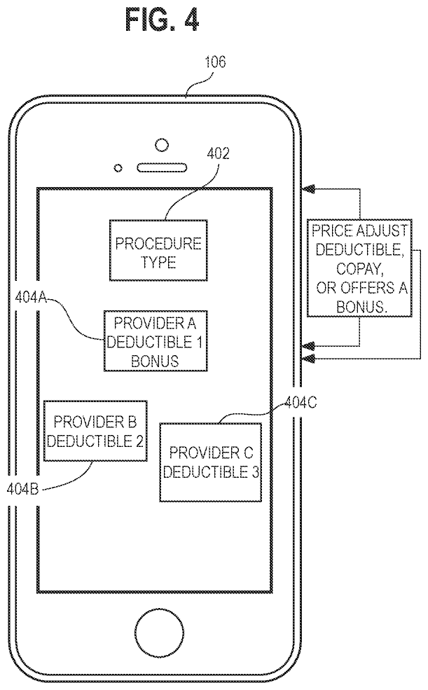

FIG. 4 illustrates an exemplary GUI of the mobile application executing on the mobile user devices 106 displaying the adjusted premium, deductible, or incentive. The exemplary GUI includes a procedure type element 402 and a plurality of provider elements 404a, 404b, 404c. The procedure type element 402 displays a name or identifier of the procedure (e.g., “knee replacement”, etc.). Each provider element 404a, 404b, 404c displays a name or identifier of the provider (e.g., “Provider A”, etc.) and a premium, deductible, or incentive associated with selecting the provider to perform the procedure displayed in the procedure type element 402. Although the exemplary GUI illustrated in FIG. 4 includes three provider elements 404a, 404b, 404c, those skilled in the art will appreciate that the GUI may include any number of provider elements.

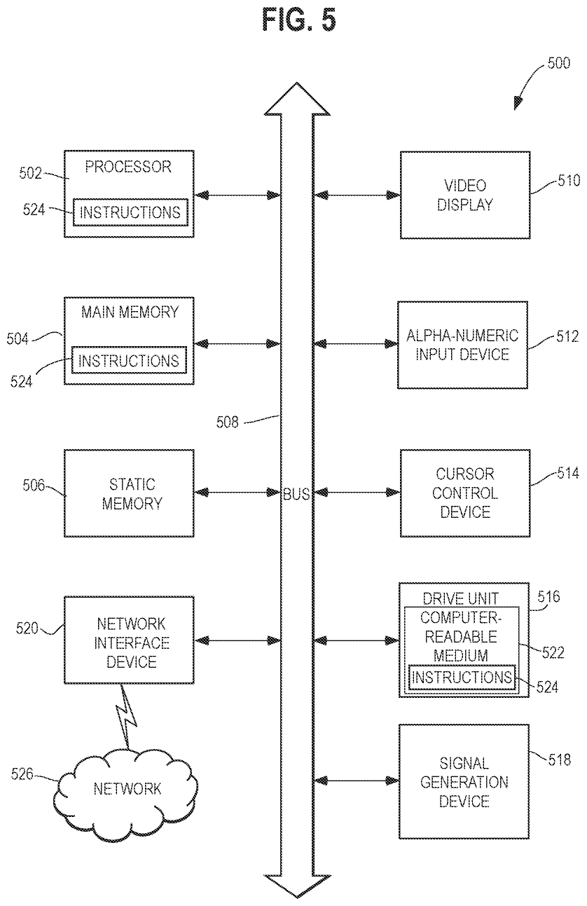

FIG. 5 is a block diagram of an example embodiment of a computer system 500 upon which embodiments of the inventive subject matter can execute. The description of FIG. 5 is intended to provide a brief, general description of suitable computer hardware and a suitable computing environment in conjunction with which the invention may be implemented. In some embodiments, the inventive subject matter is described in the general context of computer-executable instructions, such as program modules, being executed by a computer. Generally, program modules include routines, programs, objects, components, data structures, etc., that perform particular tasks or implement particular data types. The computer system 500 may comprise, in whole or in part, one or more of the healthcare provider computing device 102, the server 104, and the mobile user device 106.

The system as disclosed herein can be spread across many physical hosts. Therefore, many systems and sub-systems of FIG. 5 can be involved in implementing the inventive subject matter disclosed herein.

Moreover, those skilled in the art will appreciate that the invention may be practiced with other computer system configurations, including hand-held devices, multiprocessor systems, microprocessor-based or programmable consumer electronics, smart phones, network PCs, minicomputers, mainframe computers, and the like. Embodiments of the invention may also be practiced in distributed computer environments where tasks are performed by I/O remote processing devices that are linked through a communications network. In a distributed computing environment, program modules may be located in both local and remote memory storage devices.

With reference to FIG. 5, an example embodiment extends to a machine in the example form of a computer system 500 within which instructions for causing the machine to perform any one or more of the methodologies discussed herein may be executed. In alternative example embodiments, the machine operates as a standalone device or may be connected (e.g., networked) to other machines. In a networked deployment, the machine may operate in the capacity of a server or a client machine in server-client network environment, or as a peer machine in a peer-to-peer (or distributed) network environment. Further, while only a single machine is illustrated, the term “machine” shall also be taken to include any collection of machines that individually or jointly execute a set (or multiple sets) of instructions to perform any one or more of the methodologies discussed herein.

The example computer system 500 can include a processor 502 (e.g., a central processing unit (CPU), a graphics processing unit (GPU) or both), a main memory 504 and a static memory 506, which communicate with each other via a bus 508. The computer system 500 can further include a video display unit 510 (e.g., a liquid crystal display (LCD) or a cathode ray tube (CRT)). In example embodiments, the computer system 500 also includes one or more of an alpha-numeric input device 512 (e.g., a keyboard), a user interface (UI) navigation device or cursor control device 514 (e.g., a mouse), a disk drive unit 516, a signal generation device 518 (e.g., a speaker), and a network interface device 520.

The disk drive unit 516 includes a machine-readable medium 522 on which is stored one or more sets of instructions 524 and data structures (e.g., software instructions) embodying or used by any one or more of the methodologies or functions described herein. The instructions 524 may also reside, completely or at least partially, within the main memory 504 or within the processor 502 during execution thereof by the computer system 500, the main memory 504 and the processor 502 also constituting machine-readable media.

While the machine-readable medium 522 is shown in an example embodiment to be a single medium, the term “machine-readable medium” may include a single medium or multiple media (e.g., a centralized or distributed database, or associated caches and servers) that store the one or more instructions. The term “machine-readable medium” shall also be taken to include any tangible medium that is capable of storing, encoding, or carrying instructions for execution by the machine and that cause the machine to perform any one or more of the methodologies of embodiments of the present invention, or that is capable of storing, encoding, or carrying data structures used by or associated with such instructions. The term “machine-readable storage medium” shall accordingly be taken to include, but not be limited to, solid-state memories and optical and magnetic media that can store information in a non-transitory manner, i.e., media that is able to store information. Specific examples of machine-readable media include non-volatile memory, including by way of example semiconductor memory devices (e.g., Erasable Programmable Read-Only Memory (EPROM), Electrically Erasable Programmable Read-Only Memory (EEPROM), and flash memory devices); magnetic disks such as internal hard disks and removable disks; magneto-optical disks; and CD-ROM and DVD-ROM disks.

The instructions 524 can further be transmitted or received over a communications network 526 using a signal transmission medium via the network interface device 520 and utilizing any one of a number of well-known transfer protocols (e.g., FTP, HTTP). Examples of communication networks include a local area network (LAN), a wide area network (WAN), the Internet, mobile telephone networks, Plain Old Telephone (POTS) networks, and wireless data networks (e.g., WiFi and WiMax networks). In an embodiment, communications network 526 corresponds to communications network 108. The term “machine-readable signal medium” shall be taken to include any transitory intangible medium that is capable of storing, encoding, or carrying instructions for execution by the machine, and includes digital or analog communications signals or other intangible medium to facilitate communication of such software.

From the foregoing, it will be seen that this invention is one well adapted to attain all the ends and objects hereinabove set forth together with other advantages which are obvious and which are inherent to the structure. It will be understood that certain features and sub combinations are of utility and may be employed without reference to other features and sub combinations. Since many possible embodiments of the invention may be made without departing from the scope thereof, it is also to be understood that all matters herein set forth or shown in the accompanying drawings are to be interpreted as illustrative and not limiting.

The constructions described above and illustrated in the drawings are presented by way of example only and are not intended to limit the concepts and principles of the present invention. Thus, there has been shown and described several embodiments of a novel invention. As is evident from the foregoing description, certain aspects of the present invention are not limited by the particular details of the examples illustrated herein, and it is therefore contemplated that other modifications and applications, or equivalents thereof, will occur to those skilled in the art. The terms “having” and “including” and similar terms as used in the foregoing specification are used in the sense of “optional” or “may include” and not as “required”. Many changes, modifications, variations and other uses and applications of the present construction will, however, become apparent to those skilled in the art after considering the specification and the accompanying drawings. All such changes, modifications, variations and other uses and applications which do not depart from the spirit and scope of the invention are deemed to be covered by the invention.

What is claimed is:

1. A system, comprising:
an online tool executing on a server computing device, wherein the online tool is operable to:
receive a change to a price of a healthcare procedure from a healthcare provider computing device; and
dynamically adjust in real-time a deductible, premium, or incentive for an insurance plan of at least one patient covering the healthcare procedure based on the received changed price.

2. The system of claim 1, wherein the online tool is further operable to transmit data comprising the changed price to a mobile application executing on a mobile user device of the at least one patient.

3. The system of claim 2, wherein the mobile application is configured to display a healthcare procedure type, at least one healthcare provider associated with the healthcare procedure type, and a price associated with the healthcare procedure type for each at least one healthcare provider.

4. The system of claim 1, wherein the online tool is further operable to transmit data comprising the dynamically adjusted premium, deductible, or incentive to a mobile application executing on a mobile user device of the at least one patient.

5. The system of claim 4, wherein the mobile application is configured to display a healthcare procedure type, at least one healthcare provider associated with the healthcare procedure type, and a deductible, premium, or incentive for an insurance plan associated with the healthcare procedure type for each at least one healthcare provider.

6. The system of claim 1, wherein the healthcare provider computing device is operable to provide a menu builder to a user to allow the user to change the price of the healthcare procedure.

7. The system of claim 6, wherein the online tool is operable to transmit data comprising the change price to a mobile application executing on a mobile user device of the at least one patient in real-time.

8. The system of claim 6, wherein the menu builder operating on the healthcare provider computing device is operable to allow the user to create a new healthcare procedure and provide a price associated with the new healthcare procedure.

9. The system of claim 8, wherein the online tool is operable to receive the new healthcare procedure and the price associated with the new healthcare procedure and transmit data comprising the new healthcare procedure and the price to a mobile application executing on a mobile user device of the at least one patient.

10. The system of claim 1, wherein the server computing device and the healthcare provider computing device are communicatively coupled via a communications network.

11. A method of dynamically adjusting in real-time a deductible, premium, or incentive for an insurance plan, the method comprising the steps of:
receiving a price change of a healthcare procedure from a healthcare provider through a healthcare provider computing device;
transmitting the price change of the healthcare procedure to an online tool executing on a server computing device; and
dynamically adjusting in real-time a deductible, premium, or incentive for an insurance plan of at least one patient covering the healthcare procedure based on the price change.

12. The method of claim 11, further comprising the step of transmitting data comprising the price change from the online tool to a mobile application executing on a mobile user device of the at least one patient.

13. The method of claim 11, further comprising the step of transmitting data comprising the dynamically adjusted premium, deductible, or incentive from the online tool to a mobile application executing on a mobile user device of the at least one patient.

14. A system for dynamically adjusting in real-time a deductible, premium, or incentive for an insurance plan, the system comprising:
an online tool executing on a server computing device;
a menu builder executing on a healthcare provider computing device associated with a healthcare provider; and
a mobile application executing on a mobile user device associated with at least one patient;
wherein the online tool is operable to receive a change to a price of a healthcare procedure from the healthcare provider through the healthcare provider computing device, and dynamically adjust in real-time a deductible, premium, or incentive for an insurance plan of the at least one patient covering the healthcare procedure based on the received changed price.

15. The system of claim 14, wherein the online tool is further operable to transmit data comprising the changed price to the mobile application executing on the mobile user device of the at least one patient.

16. The system of claim 14, wherein the online tool is further operable to transmit data comprising the dynamically adjusted premium, deductible, or incentive to the mobile application executing on the mobile user device of the at least one patient.

17. The system of claim 14, wherein the server computing device, the healthcare provider computing device, and the mobile user device are communicatively coupled via a communications network.
